(19) United States
(12) Patent Application Publication
(10) Pub. No.: US 2021/0244015 A1
(43) Pub. Date: Aug. 12, 2021

(54) INSECT RELEASE DEVICE

(71) Applicant: Verily Life Sciences LLC, South San Francisco, CA (US)

(72) Inventors: Charles Behling, Brisbane, CA (US); Brian Wasson, Columbus, OH (US)

(73) Assignee: Verily Life Sciences LLC, South San Francisco, CA (US)

(21) Appl. No.: 16/787,334

(22) Filed: Feb. 11, 2020

(51) Int. Cl. A01M 99/00; A01K 67/033; B65D 43/02; B65D 47/06; B65D 51/24

(52) U.S. Cl. CPC A01M 99/00; A01K 67/033; B65D 51/242; B65D 47/06; B65D 43/0231

(57) ABSTRACT

Described herein are methods and systems for containing and releasing insects in a selective manner. An example system may include a container that defines and inner volume, the container open at one end, a lid, and a roosting panel. The lid includes a port defining a load-release pathway that interfaces with an insect sorting device and enables release of insects from within the container. The roosting panel may be coupled to the lid and extend into the inner volume when the lid is secured to the container. The roosting panel includes surface for insects to grasp onto within the inner volume.

### BACKGROUND

As part of a Sterile Insect Technique (SIT) program, insects be may be classified as male or female and selectively sterilized before being released into the wild. Such programs may be implemented to minimize or eliminate insect-borne diseases and/or to manage insect populations in certain areas. Depending on the program, classification and sterilization may be performed at one or more stages of insect development. Sterilized insects may be released in a variety of ways and using various device, ranging from hand-held devices to larger scale release mechanisms.

### BRIEF SUMMARY

A system for insect release as part of a sterile insect technique is described. One general aspect includes an insect release device, including a container defining an inner volume with a container opening at one end; and a lid to releasably engage with the container to cover the container opening and enclose the inner volume, the lid including a roosting panel coupled to the lid and positioned to extend into the inner volume when the lid is secured to the container. The roosting panel includes a surface for insects to roost upon. The insect release device also includes a port defining a load-release pathway, the port sized to interface with an insect sorting device that loads insects from the insect sorting device via the load-release pathway into the inner volume, where the load-release pathway is configured to enable release of the insects after loading of the insects.

One general aspect includes an insect release device including a cylindrical container enclosing an inner volume and defining a container opening at a first end of the cylindrical container. The insect release device also includes a first port on a lateral side of the cylindrical container and a lid to releasably engage with the container to cover the container opening and enclose the inner volume. The lid includes a second port defined within a central region of the lid, the second port sized to interface with an insect sorting device to load insects from the insect sorting device. The insect release device also includes a cylindrical member positioned to slidably interface with the second port and extend to a second end of the cylindrical container. The insect release device also includes a roosting structure within the cylindrical container, the roosting structure including a shape matching an interior of the cylindrical container. The insect release device also includes at least two roosting panels extending vertically along a length of the cylindrical container from the end support and also extending radially from the center of the cylindrical container, the at least two roosting panels and the cylindrical member dividing the inner volume into at least two distinct volumes. The insect release device also includes a rotation hub extending from the end support along the length of the cylindrical container and sized to extend through the second port when the roosting structure is within the inner volume enclosed by the cylindrical container and the lid, enabling rotation of the roosting structure within the cylindrical container. The insect release device also includes a release cap shaped to releasably seal the first port.

One general aspect includes an insect release device including a cylindrical container defining an inner volume with an opening at a first end. The insect release device also includes a lid to releasably engage with the cylindrical container to cover the opening and enclose the inner volume, the lid including; a perimeter portion; and a rotating panel rotatably coupled to the perimeter portion positioned to rotate independently of the perimeter portion. The rotating panel includes a first port positioned within a central portion of the lid. The insect release device also includes a second port positioned within an off-center portion of the lid. The insect release device also includes a cylindrical member positioned to slidably interface with the first port and extending through the inner volume to a second end of the cylindrical container; and a roosting structure, including an end support having a circular shape. The insect release device also includes at least two roosting surfaces coupled to the end support and positioned to extend into the inner volume, the at least two roosting surfaces and the cylindrical member dividing the inner volume into at least two distinct volumes.

One general aspect includes an insect release device including a primary chamber defining an inner volume, the primary chamber including a first port shaped to interface with an insect sorting device and enable loading insects from the insect sorting device and into the primary chamber. The insect release device also includes two chamber ports defined by the primary chamber, each defining a first conduit between an exterior and the inner volume. The insect release device also includes a slot defined between the two chamber ports defining a second conduit between the exterior and the inner volume. The insect release device also includes a divider including an extension positioned to slidably insert into the slot and extend a length of the primary chamber to divide the inner volume into two distinct volumes when the divider is inserted into the slot. The insect release device also includes two secondary chambers positioned to slidably couple with the two chamber ports to enclose the two distinct volumes within the two secondary chambers.

Another general aspect includes a method of releasing insects, including inserting a plurality of roosting surfaces into a container of an insect release device. The method of releasing insects also includes securing a lid to the container, thereby enclosing an inner volume with the plurality of roosting surfaces inside the inner volume. The method also includes loading insects into the release device from an insect sorting device through an introduction port. The method of releasing insects also includes sealing the introduction port of the release device. The method of releasing insects also includes opening a release port of the release device to release the insects. Other embodiments of this aspect include corresponding devices and systems each configured to perform the actions of the methods.

### BRIEF DESCRIPTION OF THE DRAWINGS

The accompanying drawings, which are incorporated into and constitute a part of this specification, illustrate one or more certain examples and, together with the description of the example, serve to explain the principles and implementations of the certain examples.

FIG. 1 illustrates a hand-held insect release device having a single port for filling and emptying the device, according to at least one example.

FIG. 2 illustrates an exploded view of the hand-held release device of FIG. 1, according to at least one example.

FIG. 3 illustrates a perspective view of the inside of the lid of the hand-held insect release device of FIG. 1, according to at least one example.

FIG. 4 illustrates a view of a roosting panel of the hand-held release device of FIG. 1, according to at least one example.

FIG. 5 illustrates a hand-held release device having a rotatable divider for selectively releasing a portion of the insects within the device, according to at least one example.

FIG. 6 illustrates the hand-held release device of FIG. 5 with a central filling tube partially removed along a filling axis to allow movement between internal sections of the device, according to at least one example.

FIG. 7 illustrates a roosting structure and divider to divide the internal volume of a hand-held release device, according to at least one example.

FIG. 8 illustrates a hand-held release device having rotatable openings on either end of the device to selectively release insects from divided sections of the internal volume of the device, according to at least one example.

FIG. 9 illustrates a hand-held release device having a rotatable internal divider and a release port on a side of the device, according to at least one example.

FIG. 10 illustrates a container portion of a hand-held release device having a port for filling and releasing insects on a side of the container, according to at least one example.

FIG. 11 illustrates a system for filling a plurality of insect release containers at the same time, according to at least one example.

FIG. 12 illustrates a method of filling an insect release container, according to at least one example.

FIG. 13 illustrates a method of releasing insects from an insect release container, according to at least one example.

### DETAILED DESCRIPTION

Examples are described herein in the context of storage and release of adult stage insects, and in particular adult stage mosquitoes. Those of ordinary skill in the art will realize that the following description is illustrative only and is not intended to be in any way limiting. For example, the techniques described herein may be used to store and release mosquitoes in other stages or other insects. Reference will now be made in detail to implementations of examples as illustrated in the accompanying drawings. The same reference indicators will be used throughout the drawings and the following description to refer to the same or like items.

In the interest of clarity, not all of the routine features of the examples described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another.

As part of a sterile insect technique (SIT) program or otherwise, it is desirable to store, transport, and release known quantities of insects. The example systems, methods, and devices described below are used to store, transport, and release known quantities of insects using reusable containers. Each insect release device may hold hundreds, thousands, tens of thousands, or even more insects. To increase density, in some applications, insects are compressed as they are loaded into the insect release device.

The insect release devices interface with an insect sorting device or storage device as part of the SIT program and are filled with insects from the sorting device or storage device. The insect release devices allow for controlled release of insects and allows a user or SIT program to release the insects in multiple stages or locations rather than all at once. Since many insects do not travel far from their release locations, this allows for greater geographic coverage of the SIT program using fewer release containers. Hand releasing insects from an insect release device may result in insects being spread over a large geographic area. The insect release devices may be used to drive insects out of inner volumes, speeding up the release of insects. The insect release devices described below also included options for feeding and roosting insects so they may be stored in the release devices for greater periods of time than in some previous systems.

The insect storage and release systems described herein may be shipped to end users for release. For example, a homeowner participating in a SIT program may receive, in the mail or otherwise, an insect storage and release system including a set number of insects and instructions for releasing the insects (e.g., release one section of the storage and release system per day for a week). The homeowner may be responsible for opening each section and releasing the insects. For example, for a typical yard, about two sections including 1000 mosquitoes may be opened each week.

In an example, an insect release device has a container and a lid to enclose insects that are stored, transported, and subsequently released. A port on the container or the lid of the container may be used to introduce insects to the interior of the insect release device. The port may be located in the center of the lid. In some examples a second port may be off-center of the lid for releasing insects from compartments within the container. The ports may interface with an insect sorting device, as described below, and the ports may be selectively sealable. For example, the ports may include a cover or plug to close the port. The ports may have a circular opening or any other geometric cross-section. The ports may also serve as a release port for the insects as well as a port for inserting a divider to couple with a roosting panel to divide the interior of the insect release device into multiple smaller components. The roosting panel may be coupled to the lid or may be a distinct structure that includes fins or panels that extend the length and width (or radius or diameter) of the interior of the container. A middle portion of the roosting panel may be open to receive the divider and to allow free movement of insects around the interior of the container until the divider is inserted.

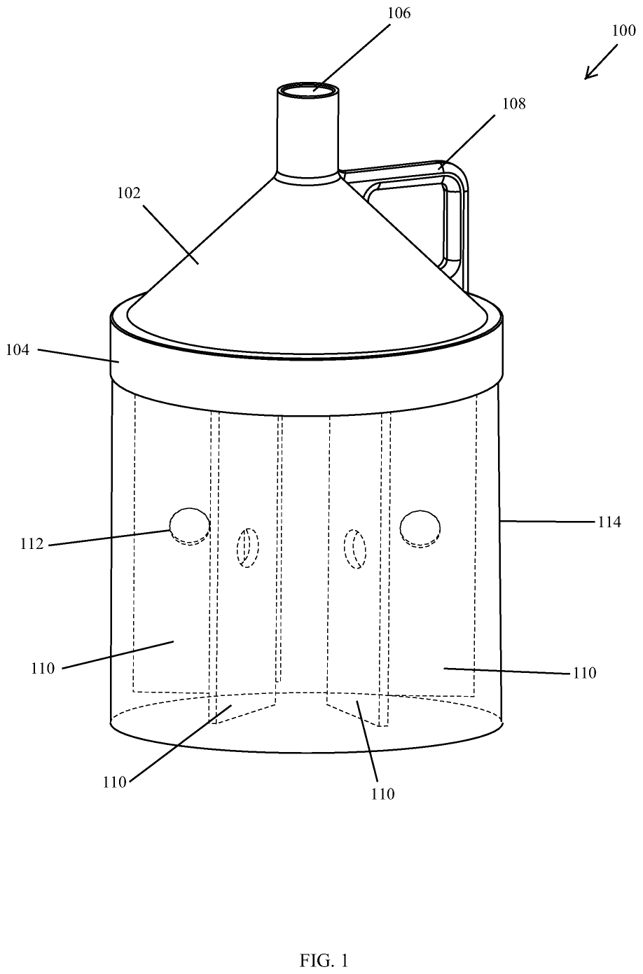

Turning now to the Figures, FIG. 1 illustrates a hand-held insect release device 100 having a single port 106 for filling and emptying the hand-held insect release device 100, according to at least one example. Insects are loaded into and released from the hand-held insect release device 100 through the single port and while inside the hand-held insect release device are contained within a single inner volume including roosting panels 110 for the insects to grasp onto. The hand-held insect release device 100 can be filled with insects for releasing as part of a sterile insect technique and also selectively release the insects. The hand-held insect release device 100 includes a container 114 and a lid 102 that couple together to enclose a volume within the hand-held insect release device 100 where the insects may be stored for transportation and subsequent release.

The lid 102 defines the single port 106 for both filling and releasing the hand-held insect release device 100. The single port 106 may be sealable with a plug, screw cap, adhesive seal, or other sealing device to shut off the single port 106. The single port 106 may be shaped and sized to interface with an output of an insect sorting device (not shown). For example, the insect sorting device may have an output conduit having a diameter of between one and several centimeters (less than half an inch to over an inch). The single port 106 may have the output of the insect sorting device inserted into the single port 106 to enable transfer of insects from the insect sorting device to the hand-held insect release device 100. The output may otherwise couple to the single port 106 so as to provide a leak-free conduit for the insects to pass through. The lid 102 also includes a handle 108 which may be grasped by a user when transporting or releasing insects. The handle 108 may be large enough for a single finger of a user or large enough for an entire hand of the user.

The lid 102 releasably couples to the container 114 at coupling interface 104. The coupling interface 104 may include a threaded interface, with threads on the lid 102 and matching threads on the container 114. In some examples, the coupling interface 104 may include other coupling interfaces, such as a lip, a detent, a deformable connection, pin and groove connections, or any other releasably securable connection type.

The container 114 is a cylindrical container defining an interior volume and an opening where the lid 102 couples to enclose the inner volume of the hand-held insect release device 100. In some examples, the container 114 may have a shape other than cylindrical, such as a rectangular prism, or any other three-dimensional shape. The container 114 may have smooth side walls or may include a texture, similar to the texture described with respect to the roosting panels 110. For example, the container 114 may include a plurality of holes through the walls, the diameter of the holes smaller than a diameter of an insect (e.g., less than one millimeter in diameter). In some examples, the inner surface of the container 114 may have grooves or bumps provided on it for insects to grasp onto. For example, shallow grooves or scratches may be provided by scoring the inner wall of the container or forming the container 114 to have a rough inner surface.

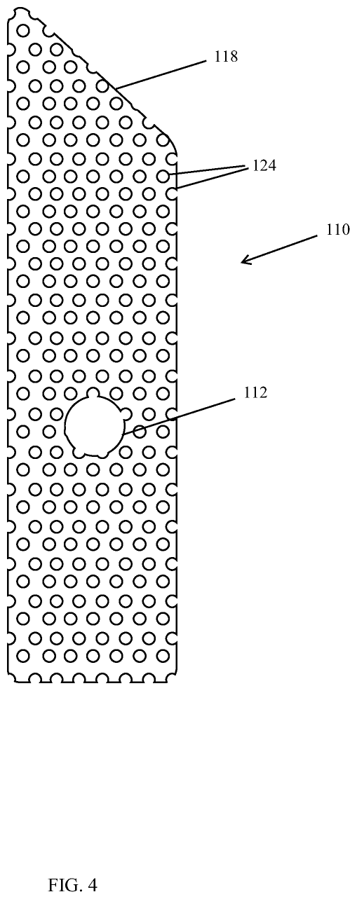

The lid 102 includes a number of roosting panels 110 that extend into the container 114. As illustrated in more detail in FIG. 4, the roosting panels 110 may have a texture other than a smooth surface, such as bumps, divots, perforations, grooves, protrusions, etc., onto which insects may grasp when inside the hand-held insect release device 100. The roosting panels 110 also define feeding openings 112. The feeding openings 112 may receive and retain insect food, for example on a porous substrate that is inserted into the feeding opening 112. The roosting panels 110 may be permanently connected to the lid 102 or may be removable from the lid 102 at an underside of the lid 102. In some examples, the roosting panels 110 may be independent of the lid 102 and the container 114 and be a separate insert that rests between the lid 102 and the container 114. There may be one or any suitable number of roosting panels 110 within the hand-held insect device 100. In some examples, there may be six roosting panels 110 on which the insects may rest when within the hand-held insect release device 100. The edges of the roosting panels 110 may extend and contact the container 114. In some examples there may be a gap of a few millimeters between the roosting panels 110 and the container 114 to enable movement of insects around the roosting panels 110.

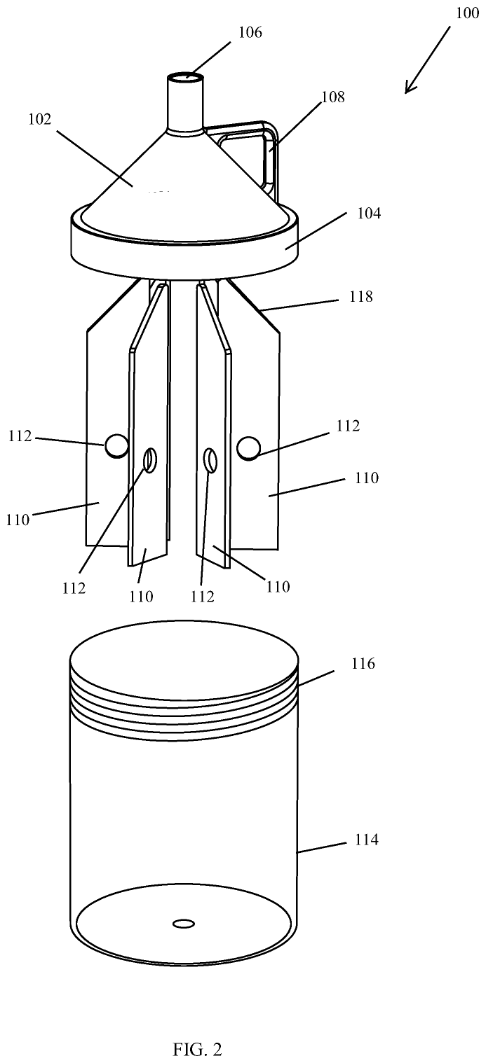

FIG. 2 illustrates an exploded view of the hand-held insect release device 100 of FIG. 1, according to at least one example. The exploded view shows a threaded interface 116 of the container 114. The threaded interface 116 engages with the coupling interface 104 on the lid. The hand-held insect release device 100 is shown disassembled and can be assembled by connecting the roosting panels 110 to the lid 102. The lid 102 is then threaded onto the container 114 to enclose a volume within the hand-held insect release device 100. The example shown in FIG. 2 may release an entire volume of insects when opened. In some examples, such as shown and described below, different regions of insect release devices may be released at different times or through different ports without releasing all of the insects at one time through a single port that is also used to fill the hand-held insect release device 100 as shown and described in FIGS. 1 through 3.

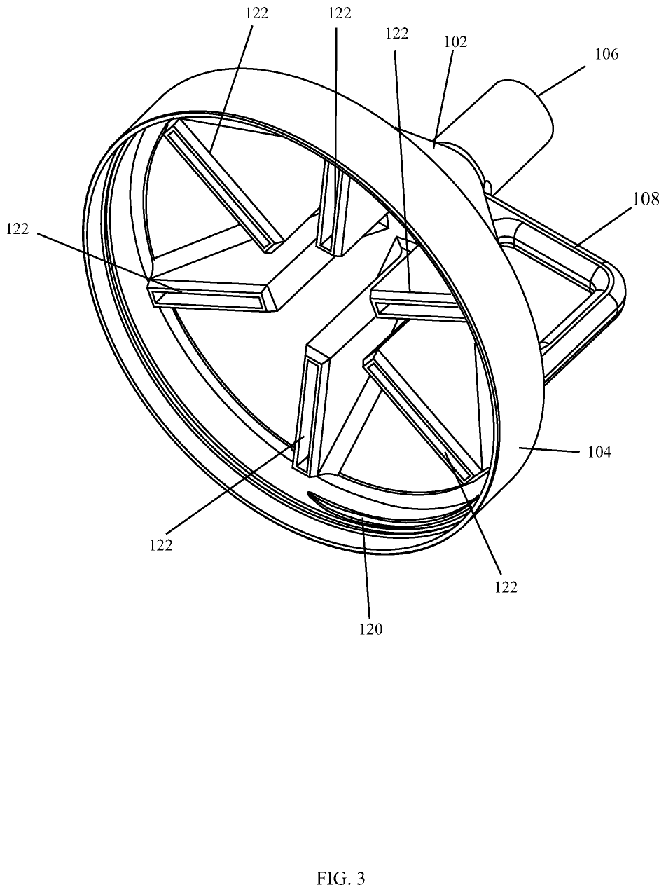

The roosting panels 110 are shown with tapered ends 118 that are insertable into slots on the underside of lid 102, as illustrated in FIG. 3. The roosting panels 110 are removable for filling the feeding openings 112 and inserting into the slots before assembling the hand-held insect release device 100. And while the roosting panels have tapered ends 118 in this example, roosting panels according to other examples may have ends with different shapes to engage with the lid. Further, the shapes of the ends may depend on the configuration of the lid 102, e.g., an example using a flat lid may employ roosting surfaces with flat upper surfaces to engage with the lid. In some examples, the ends 118 may be curved, include a releasable interface such as a snap-fit connection to connect the roosting panels 110 to the lid 102, the ends 118 may also include pins, slots, grooves, or other such features to interface with features of the lid 102.

FIG. 3 illustrates a perspective view of the inside of the lid 102 of the hand-held insect release device 100 of FIG. 1, according to at least one example. The underside of the lid 102 is shown with a threaded interface 120 of the coupling interface 104 that threads onto the threaded interface 116 of the container 114.

The underside of the lid 102 also includes the slots 122 into which the roosting panels 110 are inserted. The roosting panels 110 may be releasably or fixedly held within the slots 122 using a friction fit, pins, T-slots, grooved connections, screws, nuts and bolts, and any other suitable device. In some examples, the roosting panels 110 may be fixedly held within the slots 122 or may be integrally formed as a part of the lid structure. As illustrated in FIG. 4, the slots 122 are shaped to receive the ends 118 of the roosting panels 110. As described above, the slots 122 may have a tapered, curved, or other such shape to couple with the ends 118 to account for the shape of the lid 102, which tapers from the width of the container 114 to the width of the single port 106. In some examples, the slots 122 may have other shapes and may include snap-fit, slots, grooves, or other features for releasably securing the ends 118 to the lid 102.

FIG. 4 illustrates a view of a roosting panel 110 of the hand-held insect release device 100 of FIG. 1, according to at least one example. The roosting panel 110 is shown with the tapered end 118 and the feeding opening 112 described previously. The roosting panel 110 is shown with one example of a texture to which insects can grasp. The texture of roosting panel 110 is provided by a series of small openings 124. The small openings 124 may be smaller than one millimeter or may be up to several millimeters in diameter. The small openings 124 may provide additional surface area as well as edges to which the insects can grasp.

In some other examples, the texture of the roosting panel 110 may be provided by other textures or patterns. For example, the surface of the roosting panel 110 may be scored or grooved in parallel rows or in cross-hatching to provide edges for insects to grasp. In addition to grooves or scored marks in the surface of the roosting panel 110, the roosting panel 110 may include protrusions, such as small bumps or finger-like extensions extending perpendicularly from the surface of the roosting panel 110. The roosting panel 110 may be 3D printed to provide a texture for the insects to grasp onto. In some examples, the roosting panel 110 may not be a solid panel, but may be a wireframe or porous structure.

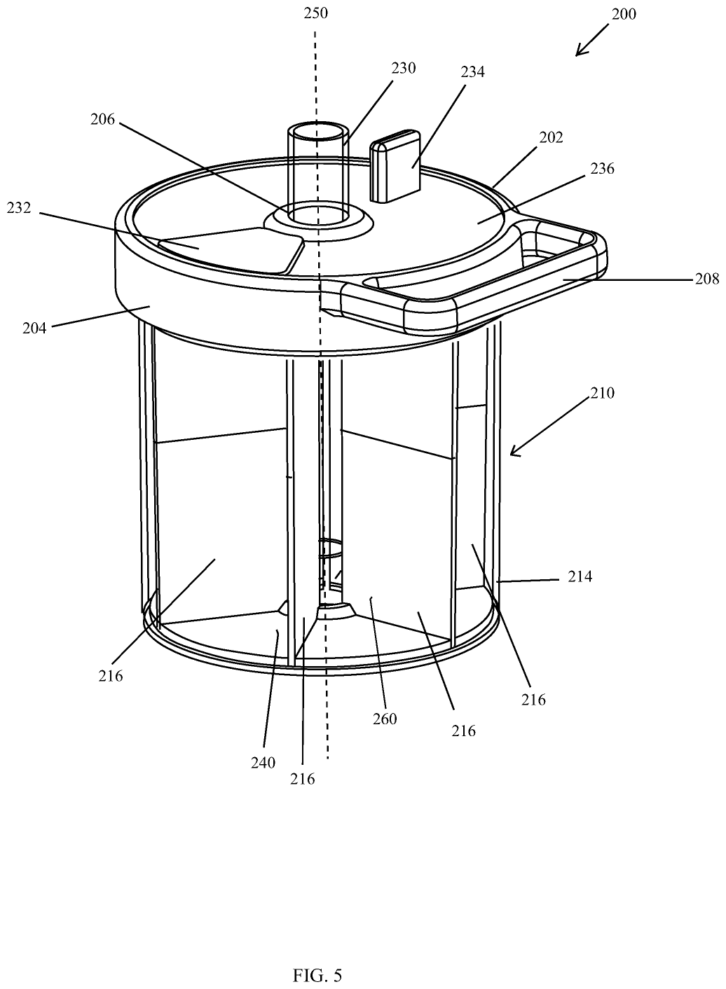

FIG. 5 illustrates a hand-held insect release device 200 having a rotatable divider 210 for selectively releasing a portion of the insects within the hand-held insect release device 200, according to at least one example. The hand-held insect release device 200 includes a container 214 and a lid 202 as well as the rotatable divider 210. The container 214 may be the same as the container 114 described above.

Rather than including only a single opening, the hand-held insect release device 200 includes multiple openings for alternative manners of filling and releasing insects. In addition, the inner volume may be divided into a number of smaller volumes 260 which can be selectively released to control the number of insects released at any particular time or location.

The lid 202 includes a securable connection 204 at the perimeter of the lid 202 the releasably connect to the container 214 such as a threaded interface, pin in groove, detent, or other releasable connection. The lid 202 has an insect port 206 at the center of the lid 202 that provides a conduit between the outside of hand-held insect release device 200 and an inner volume within the hand-held insect release device 200. The insect port 206 may be used for filling or releasing, or both filling and releasing insects from the hand-held insect release device 200 in a similar manner as the single port 106 described above with respect to FIG. 1. The insect port 206 is at the center of the lid 202 aligned with a center axis 250 that is perpendicular to the lid 202 and passes through the center of the lid 202. The lid 202 also includes a handle 208 for grasping when transporting or releasing insects from the hand-held insect release device 200.

The lid includes a rotating plate 236 defining an upper portion of lid 202. The rotating plate 236 is rotatably secured with the outer perimeter of lid 202. The outer perimeter of the lid 202 includes the securable connection 204 as well as a groove which the rotating plate 236 rests within. The rotating plate 236 within the groove is free to rotate with respect to the outer perimeter of the lid 202. The rotating plate 236 includes a pin 234 and a port cover 232. The pin 234 extends from the rotating plate 236 and may be grasped by a user to spin the rotating plate 236 with respect to the outer perimeter of lid 202. The port cover 232 is offset from the center of the rotating plate 236. As the rotating plate 236 spins within the groove of the outer perimeter of lid 202, the port cover 232 is moved around the middle portion of the lid 202. When the port cover 232 is removed, an opening is uncovered that allows insects to be inserted or released.

The rotatable divider 210 rests within the interior volume of the hand-held insect release device 200. The rotatable divider 210 includes a disk 240 at a base of the rotatable divider 210 with a number of panels 216 extending perpendicular to the disk 240 towards the lid 202. The panels 216 may be similar to the roosting panels 110 described above with respect to FIGS. 1 through 4. The panels 216 may include a textured surface for insect roosting and may also include feeding ports. At an opposite end of the panels 216 from the disk 240 (e.g., at the base of the rotatable divider 210), a second ring may couple the ends of the panels 216 together. The panels 216 of the rotatable divider 210 define a central passage along the center axis 250. In this manner, the rotatable divider 210, including the panels 216, is rotatable within the interior volume of the hand-held insect release device 200.

A cylindrical divider 230 slidably interfaces with the insect port 206 to move in an out of the lid 202. The cylindrical divider 230 is removable from the hand-held insect release device 200. The cylindrical divider 230 may be removed for filling the hand-held insect release device 200 and then re-inserted into the hand-held insect release device 200 to close different regions within the hand-held insect device 200 as described below. The cylindrical divider 230 may be partially removable such as sliding halfway out of the hand-held insect release device 200, or may be fully removable. To fill the hand-held insect release device 200, the cylindrical divider 230 may be removed so each of the smaller volumes within the container 214 are in fluid communication with each other. Insects may be introduced from the outlet of an insect sorting device or other insect rearing or storage device into the insect port 206. After the insects are added the outlet of the insect sorting device may be removed and the cylindrical divider 230 inserted into the insect port 206 to divide the inner volume of the hand-held insect release device 200 into smaller volumes 260 and separate the insects into a discrete number of groups based on the number of smaller volumes.

The cylindrical divider 230 may be solid or hollow and may serve to block or plug the insect port 206. The cylindrical divider 230 may also interface with longitudinal interior edges of the panels 216 of the rotatable divider 210 to divide the inner volume of the hand-held insect release device 200 into a number of smaller volumes 260. Each smaller volume 260 is defined by the cylindrical divider 230, the rotatable divider 210, the lid 202, and the container 214. The panels 216 of the rotatable divider 210 interface with an interior surface of the wall of the container 214 to form the smaller volumes 260. The rotatable divider 210 may contact the inner wall of the container 214 or may leave a gap of less than one millimeter between the rotatable divider 210 and the container 214. In some examples the gap may be larger, such as several millimeters or large enough for insects to travel freely between adjacent smaller volumes 260. The cylindrical divider 230 extends the full length of the hand-held insect release device 200 from the insect port 206 of the lid 202 to the base of container 214.

As the rotating plate 236 spins with respect to the lid 202, the port cover 232 moves and provides a conduit between each of the smaller volumes 260 defined by the cylindrical divider 230, the rotatable divider 210, the lid 202, and the container 214 and the exterior of the hand-held insect release device 200. To release insects from the hand-held insect release device 200, the port cover 232 is opened so that insects in a first smaller volume are free to move from the smaller volume via the port covered by port cover 232 to outside the container 214. After the insects from the first smaller volume are released, the rotating plate 236 is spun by a user grasping the pin 234. The port cover 232 is then aligned with a second smaller volume and insects contained inside the second smaller volume may be released through the port once the port cover 232 is removed. This same process may be repeated to selectively release insects from each of the small volumes.

In some examples, insects may be introduced into the smaller volumes 260 through the port covered by port cover 232. The port provides a conduit or direct access between a particular smaller volume 260 and the exterior of the insect release device 200. The port may be used to introduce a precise number of insects into each of the smaller volumes 260. The rotatable divider 210 may be rotated after loading insects into each smaller volume to access another smaller volume.

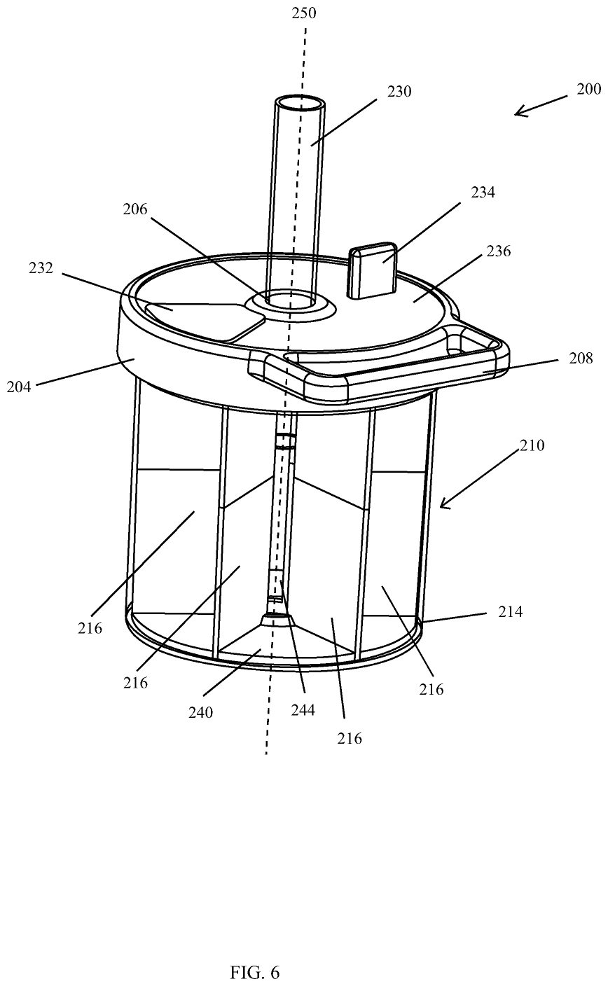

FIG. 6 illustrates the hand-held insect release device 200 of FIG. 5 with the cylindrical divider 230 partially removed to allow movement between internal sections of the hand-held insect release device 200, according to at least one example. As described above, the cylindrical divider 230 when partially removed allows free movement of insects between the smaller volumes of the interior. In a space 244 between the bottom of the cylindrical divider 230 and the bottom of the container, there is an open conduit between each of the smaller volumes. The space 244 may enable insects to move between different areas of the inner volume or between smaller volumes 260. Each smaller volume 260 may then be divided from one another to stop movement of insects between smaller volumes by sliding the cylindrical divider 230 fully into the hand-held insect release device 200.

As insects are added to the hand-held insect release device 200, they will naturally distribute evenly throughout the inner volume. In some examples, when food is added to each of the smaller volumes, the insects may separate into discrete groups in each smaller volume of roughly equivalent numbers. After the cylindrical divider 230 is inserted, the insects may be released from each smaller volume based on a number of insects to be released. For example, a total of 600 insects may be added to the hand-held insect release device 200. The 600 insects may divide themselves roughly evenly between six smaller volumes, each containing roughly 100 insects. When the insects are to be released, a user may release one, two, three, or more sections of the hand-held insect release device to release 100, 200, 300, or more insects in a particular geographic area.

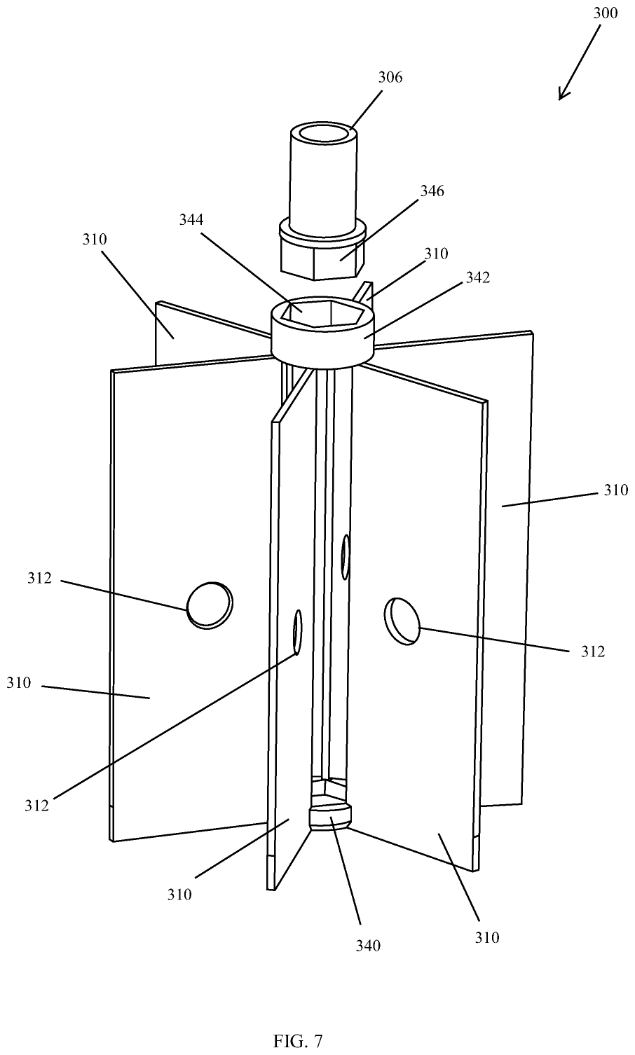

FIG. 7 illustrates an alternative roosting structure 300 that also divides the internal volume of a hand-held insect release device, such as the hand-held insect release device 200 of FIGS. 5 and 6 described above, according to at least one example. The roosting panels 310 may contact the container 214 and interface with a cylindrical divider 230 to define the smaller volumes. Each end of the roosting structure may not have a disk 240 as shown in FIGS. 5 and 6. The roosting structure 300 includes roosting panels 310 similar to the roosting panels 110 described above, including feeding ports 312 and a texture for insects to grasp onto. The roosting panels 310 couple to a first ring 340 and a second ring 342 at opposite ends of the roosting panels 310. The first ring 340 and the second ring 342 position and maintain the roosting panels 310 to divide the interior of the hand-held insect release device 200 into six equal compartments. In some examples the roosting structure 300 may divide the interior into less than or more than six compartments or smaller volumes. A cylindrical tube or other similar structure may extend from the first ring 340 to the second ring 342 in place of the cylindrical divider 230 described above to define the smaller volumes 260.

The second ring 342 also defines a port 344 through which insects may be introduced and which may also be used to rotate the roosting structure 300. The port 344 may have a non-circular profile, such as a hexagonal profile so a rotational torque may be applied to the roosting structure to rotate it within the container 314. The roosting structure 300 may be used with a hand-held insect release device having a lid similar to lid 202 where the rotating panel 236 may be fixed in a single position and by rotating the roosting panel 310, each of the smaller volumes may be released through the port covered by port cover 232. Additionally, rotating the rotating panel 236 allows insects to be introduced into each of the smaller volumes defined by the roosting panels 310 as well as the cylindrical divider 230, container 214, and lid 202.

A rotation tool 346 may be used to apply a rotational torque to the roosting structure 300 as described above. For example, the rotation tool 346 may have a mating surface or profile to interface with the port 344 and be used to apply a torque from the rotation tool 346 to the roosting structure 300 to cause the roosting structure 300 to rotate about a rotational axis that extends axially through a center of the port 344, the first ring 340, and the second ring 342. The rotation tool 346 may also include an insect port 306 for loading insects from an insect sorting device. In some examples, the rotation tool 346 may block port 344 to seal the opening.

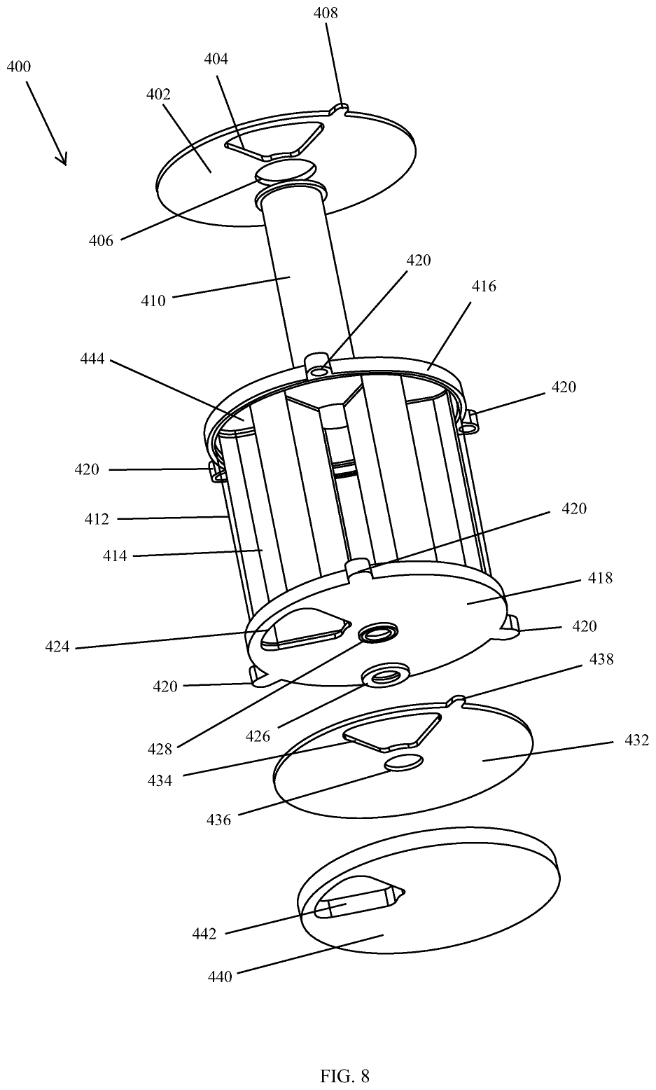

FIG. 8 illustrates a hand-held insect release device 400 having rotatable openings on either end of the device to selectively release insects from divided sections of the internal volume of the device, according to at least one example. The hand-held insect release device 400 may be filled with insects or release insects through one of four different ports. Additionally, with openings at either end of the hand-held insect release device 400, insects may be blown or forced out of the inner volume by pushing air through from one opening to another. The hand-held insect release device 400 includes a container 412 similar to the container 114 but with openings on each end. Within the container 412 is a roosting structure 414 similar to the roosting structure 300 or roosting structure 210. The hand-held insect release device 400 may be opened at opposite ends to release insects through both ends or to drive insects out of the hand-held insect release device 400 such as with driven air.

At each end, the roosting structure 412 is retained within the container 412 by a first retainer 418 and a second retainer 416. The first retainer 418 and the second retainer 416 each maintain the roosting structure 414 within the container 412 and define a port 424 and a port 444 which aligns with a section defined by the roosting structure 414. The first retainer 418 and the second retainer 416 also include retention devices 420 that couple together with a pin (not shown) or other coupler to keep the retainers in place. A central port 428 and second central port 406 provide access for filling and rotating the roosting structure 414 as described above.

At each end, a rotating plate 432 and a second rotating plate 402 are rotatably coupled to the first retainer 418 and the second retainer 416 to selectively close off port 424 and port 404. A washer 426 may be placed between the first retainer 418 and the rotating plate 432 to reduce friction between the two as the rotating place 432 rotates relative to the first retainer 418. Port 434 allows passage through the rotating plate 432 when the port 434 and the port 424 are aligned. An extension 438 of the rotating plate 432 allows a user to selectively rotate the rotating plate 432. Finally, a cover 440 at the end encloses the rotating plate 432 and the first retainer 418. At the opposite end of the hand-held insect release device an identical structure exists (though not shown) with an exception of filling tube 410. Filling tube 410 may be used to connect to an insect sorting device and introduce insects into the hand-held insect release device 400. The filling tube 410 may enable insects to flow through a center section of the filling tube and into the inner volume of the hand-held insect release device 400. Filling tube 410 may have a collar at one end to prevent it from being fully removed from the insect release device 400 but may still move axially to open a gap and enable movement between divided regions of the inner volume, as such it may serve a similar function to the cylindrical divider 230 as described above.

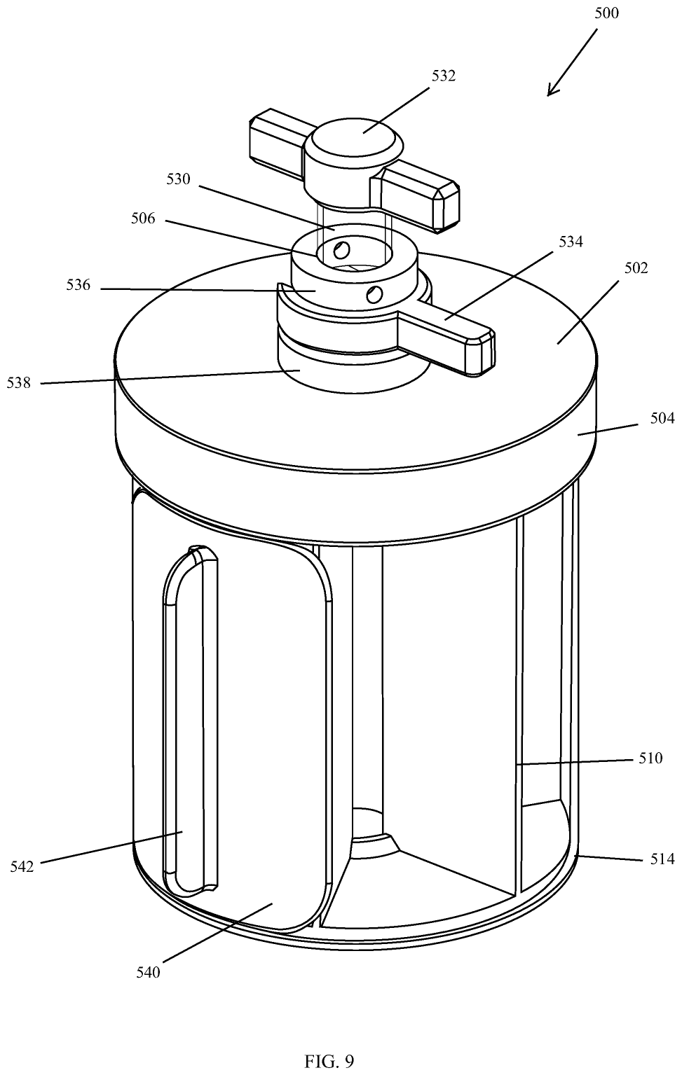

FIG. 9 illustrates a hand-held insect release device 500 having a rotatable internal divider 510 and a release port cover 540 on a side of the device, according to at least one example. Rather than rotating the cap or lid of the hand-held insect release device 500 to release insects from different smaller volumes, an internal divider within the container may be rotated to present distinct inner volumes to a release location at the side of the hand-held insect release device 500. The release port cover 540 seals and closes a port that enables introduction as well as release of insects from the insect release device 500. The rotatable internal divider 510 is an example of the roosting structure 210 described above with respect to FIGS. 5 and 6. The lid 502 may likewise be similar to the lid 102 and the lid 202 described above. The container 514 is a cylindrical container with a release port cover 540 on a side of the container 514 for releasing insects from smaller volumes divided by the rotatable internal divider 510.

The rotatable internal divider 510 is similar to the roosting structure 210 described above in including a number of panels that extend radially from a center axis of the container and has a bottom ring connecting each of the panels. The rotatable internal divider 510 is rotated using a rotation tool that includes a handle 534 that can be used to apply a torque to the rotatable internal divider 510. The rotatable internal divider 510 is rotated in a manner similar to the rotation tool 346 described above that extends above the top of the rotatable internal divider 510. The rotating insert couples with the handle 534 that is used to apply a torque to rotate the rotatable internal divider 510. The handle 534 rests on a hub 538 through which the rotating insert passes to couple with the handle 534. A second hub 536 retains the handle 534 in a captured position such that it is only rotatable in a single plane, allowing rotation of the rotatable internal divider 510. The second hub also defines the port 506 which may be used to fill the hand-held insect release device 500 similar to the port 106 described above.

A cylindrical tube 530 and cap 532 insert into the port 506 to block the port 506 and seal the port after filling with insects. Additionally, the cylindrical tube 530 interfaces with the rotatable internal divider 510 to divide the internal volume of the hand-held insect release device 500 into discrete smaller volumes as described above.

To release insects from the hand-held insect release device 500, insects are first loaded through the port 506. The cylindrical tube 530 is then inserted to divide the insects into the discrete volumes described previously. The port cover 540 is removed by pulling on handle 542 to release insects from a first one of the discrete smaller volumes. After the insects are released from the first smaller volume, the handle 534 may be rotated to apply a torque to the rotatable internal divider 510 and move a second smaller volume in place to be in communication with the release port cover 540.

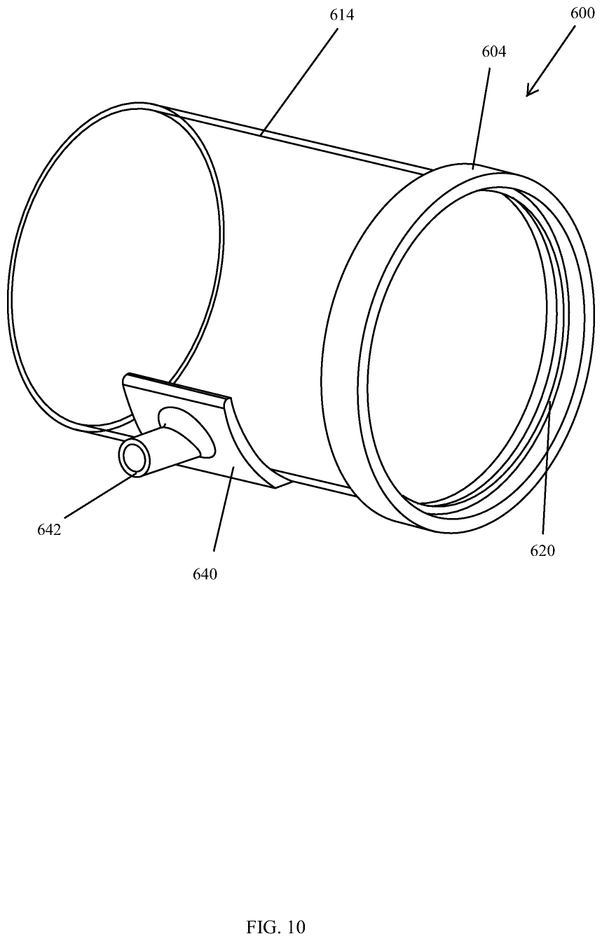

FIG. 10 illustrates a container portion 600 of a hand-held release device having a port 642 for filling and releasing insects on a side of the container, according to at least one example. The container portion 600 includes a cylindrical container 614 similar to the container 514 above. An opening 604 of the cylindrical container 614 includes a threaded interface 620 for sealing the cylindrical container 614 after inserting a roosting structure or food for insects. The lid may be a simple lid which does not have any port or passages therein.

On a side of the cylindrical container 614 is a fill and release port 642. The fill and release port 642 may be shaped and sized to interface with the outlet of an insect sorting device as with port 106 described above. The fill and release port 642 may extend from a port base adapter 640 that couples to a wall of the cylindrical container 614 similar to the release port cover 540 described above. Insects may therefore be added and released from the container through the fill and release port 642 as with other embodiments described herein. The fill and release port 642 is tangent to the cylindrical container 614. The release port 642 may be used to introduce insects into the cylindrical container 614 without the insect colliding with a perpendicular surface and potentially becoming damaged. This tangent configuration may allow insects to be introduced more rapidly with forced air or other such forcing mechanisms to quickly fill an insect release device.

The fill and release port 642 may also serve as a feeding port for insects contained within the cylindrical container 614. For example, the fill and release port 642 may provide a conduit into the interior of the cylindrical container 614 for food, such as liquids (sugared water as an example), while not allowing insects to escape. The fill and release port 642 may have a mesh covering the opening of the fill and release port 642 such that liquids or small particles of food for the insects may pass through the mesh while the insects are not allowed to escape. Though only one fill and release port 642 is shown in FIG. 10, there may be additional fill and release ports 642 on the side wall of the cylindrical container 614 as well as on the top surface or bottom surface of an insect release device.

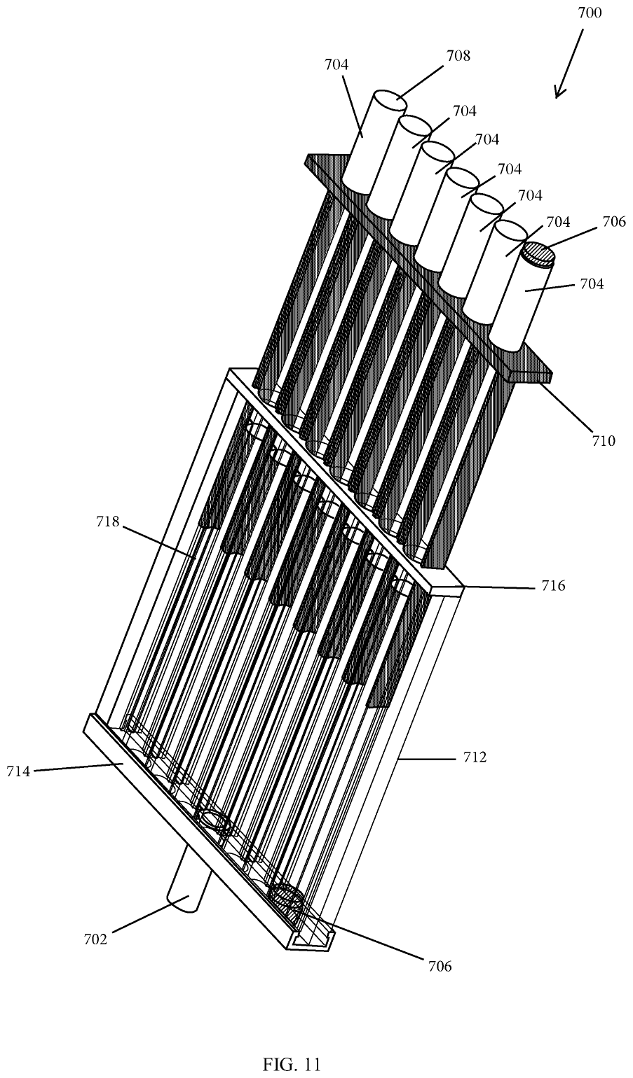

FIG. 11 illustrates a system 700 for filling a plurality of insect release containers 704 at the same time, according to at least one example. The system 700 may be used to fill a number of small volumes with insects at the same time that may then be dispersed for insect release in different locations. In this example, a number of insect release containers 704, which are cylindrical bodies, may be filled with insects simultaneously. The ends of the insect release containers may be plugged with stoppers 706. The system 700 receives insects from an insect storage or sorting device through inlet 702 which provides a passageway into an internal volume of a loading device 712.

A loading device 712 having a rectangular solid shape includes a number of openings at a first end 716 to receive the insect release containers 704 at one end. The openings slidably receive the insect release containers 704 to close the loading device 712. Within the loading device 712 is a volume that is enclosed by the insect release containers 704 when inserted into the loading device 712. The insect release containers 704 slide along the length of the loading device 712 to an end 714 of the loading device 712 where a number of stoppers 706 may be pre-installed to close the open end of the insect release containers 704.

The loading device 712 includes a number of grooves that run parallel to the passages for the insect release containers 704. A divider 710 may slide into the loading device 712 from the same end as the openings for the insect release containers 704. The divider 710 includes a base and a number of fingers or extensions that fit into the loading device and along the length of the inner volume of the loading device 712 to divide the inner volume of the loading device into distinct volumes.

To fill the insect release devices 704, insects are inserted into the loading device 712 through the port 702. The insects will then distribute themselves within the inner volume of the loading device 712. The divider 710 is inserted into the loading device 712 to divide the inner volume into distinct smaller volumes, each with insects therein. The insect release containers 704 are then inserted and slid along the length of the loading device 712 until they contact pre-installed stoppers 706 which seal the insect release containers 704. The insect release containers 704 may then be removed after enclosing and sealing the insects from each distinct volume created by the divider 710. The insect release containers 704 may then be transported to a release location where the stopper 706 may be removed to release the insects.

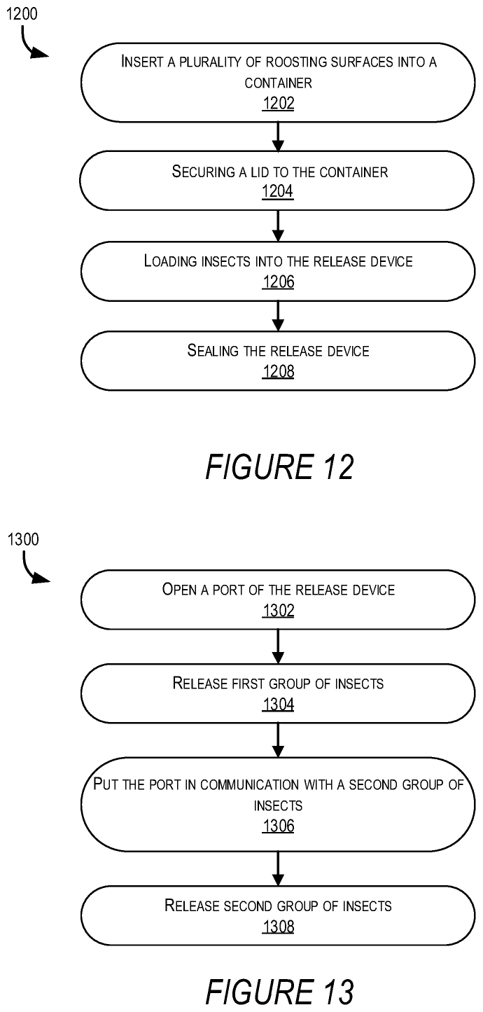

FIGS. 12 through 13 illustrate example flow diagrams showing processes 1200 and 1300 according to at least a few examples. These processes, and any other processes described herein, are illustrated as logical flow diagrams, each operation of which represents a sequence of operations that can be implemented by a user, in hardware, computer instructions, or a combination thereof.

FIG. 12 illustrates a method 1200 of filling an insect release container, according to at least one example. Though the processes described herein may be performed by a user, a computing device may also perform the following processes through the use of actuator (linear and rotational) controlled by the computing device.

At block 1202, one or more roosting surface are inserted into a hand-held insect release container. The roosting surface may be the roosting surfaces or panels described above. The hand-held insect release container may be a container of one of the hand-held insect release devices of FIGS. 1 through 10. In some examples, the roosting surface may be a divider that divides an internal volume of the hand-held insect release device into multiple smaller volumes, for example as shown in FIG. 11.

At block 1204, a lid is secured to the container, enclosing an insect retaining volume. This may be performed concurrently with the step of block 1202, for example with the roosting surfaces inserted into the hand-held insect release container as the lid is secured to the container. The insect retaining volume, defined by the hand-held insect release device, may be a single volume or may be divided into a number of smaller volumes as described above. The lid may include the structures of the lids described above, for example of FIG. 3, 5, 8, or 9.

At block 1206, insects are loaded into the release device. The insects may be loaded from an insect sorting device associated with a sterile insect technique program. The insects may also be loaded from an insect storage device. Loading the insects may involve opening a conduit, such as port 106, 442, or 642 as well as port cover 232 or 540 for the insects to traverse on their own or may involve driving the insects into the release device. Driving the insects into the release device may include using a gas or air to push the insects through a port into the insect release device.

At block 1208, the release devise is sealed for transportation or later release. This may involve blocking a port of the release device or inserting a plug or cylindrical tube as described above with respect to at least some of the embodiments herein.

FIG. 13 illustrates a method of releasing insects from an insect release container, according to at least one example. Though the processes described herein may be performed by a user, a computing device may also perform the following processes through the use of actuator (linear and rotational) controlled by the computing device.

At block 1302, a port of the release device is opened. The release device having previously been filled by insects according to method 1200. The release device may be any of the release devices described herein and the release port may be the same port as the port used to fill the insect release device or may be a different port entirely.

At block 1304, a first group of insects is released from the release device. The first group of insects may be contained within a section of the release device. For example, the first group of insects may be within a volume defined by the roosting structure.

At block 1306, the release port is placed in communication with a second group of insects. For example, this may include rotating a portion of the lid, as in FIG. 5, or rotating a roosting structure within the release device, as in FIG. 9.

At block 1308, the second group of insects is released. This may involve re-opening the port or completing rotation of the port or the roosting structure. The second group of insects may be allowed to freely escape or may, as with the first group of insects, be driven out of the release device, as with FIG. 8. For example, the insects may be driven out of the release device with a fan or directed air to speed release of the insects.

The specification and drawings are, accordingly, to be regarded in an illustrative rather than a restrictive sense. It will, however, be evident that various modifications and changes may be made thereunto without departing from the broader spirit and scope of the disclosure as set forth in the claims.

Other variations are within the spirit of the present disclosure. Thus, while the disclosed techniques are susceptible to various modifications and alternative constructions, certain illustrated examples thereof are shown in the drawings and have been described above in detail. It should be understood, however, that there is no intention to limit the disclosure to the specific form or forms disclosed, but on the contrary, the intention is to cover all modifications, alternative constructions and equivalents falling within the spirit and scope of the disclosure, as defined in the appended claims.

The use of the terms “a” and “an” and “the” and similar referents in the context of describing the disclosed examples (especially in the context of the following claims) are to be construed to cover both the singular and the plural, unless otherwise indicated herein or clearly contradicted by context. The terms “comprising,” “having,” “including,” and “containing” are to be construed as open-ended terms (e.g., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate examples of the disclosure and does not pose a limitation on the scope of the disclosure unless otherwise claimed. No language in the specification should be construed as indicating any non-claimed element as essential to the practice of the disclosure.

Conditional language used herein, such as, among others, “can,” “could,” “might,” “may,” “e.g.,” and the like, unless specifically stated otherwise, or otherwise understood within the context as used, is generally intended to convey that certain examples include, while other examples do not include, certain features, elements, and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example.

Disjunctive language such as the phrase “at least one of X, Y, or Z,” unless specifically stated otherwise, is otherwise understood within the context as used in general to present that an item, term, etc., may be either X, Y, or Z, or any combination thereof (e.g., X, Y, and/or Z). Thus, such disjunctive language is not generally intended to, and should not, imply that certain examples require at least one of X, at least one of Y, or at least one of Z to each be present.

Use herein of the word “or” is intended to cover inclusive and exclusive OR conditions. In other words, A or B or C includes any or all of the following alternative combinations as appropriate for a particular usage: A alone; B alone; C alone; A and B only; A and C only; B and C only; and all three of A and B and C.

Preferred examples of this disclosure are described herein, including the best mode known to the inventors for carrying out the disclosure. Variations of those preferred examples may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

The various features and processes described above may be used independently of one another, or may be combined in various ways. All possible combinations and sub-combinations are intended to fall within the scope of the present disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed examples. Similarly, the example systems and components described herein may be configured differently than described. For example, elements may be added to, removed from, or rearranged compared to the disclosed examples.

What is claimed is:

1. An insect release device, comprising:
a container defining an inner volume with a container opening at one end; and
a lid to releasably engage with the container to cover the container opening and enclose the inner volume, the lid comprising:
a roosting panel coupled to the lid and positioned to extend into the inner volume when the lid is secured to the container, the roosting panel comprising a surface for insects to roost upon; and
a port defining a load-release pathway, the port sized to interface with an insect sorting device that loads insects from the insect sorting device via the load-release pathway into the inner volume, wherein the load-release pathway is configured to enable release of the insects after loading of the insects.

2. The insect release device of claim 1, wherein the surface of the roosting panel comprises a textured surface for insects to grasp onto.

3. The insect release device of claim 1, wherein the lid comprises six roosting panels arranged radially on the lid.

4. The insect release device of claim 1, wherein:
the roosting panel comprises a tab at one edge; and
the lid comprises a slot to releasably secure the tab of the roosting panel to the lid.

5. The insect release device of claim 1, wherein the lid comprises a conical shape extending from a releasable connection at a base of the conical shape to the port at a top of the conical shape.

6. The insect release device of claim 1, wherein the roosting panel comprises a first edge profile that matches an inner profile of the container and extends a full length of the container.

7. The insect release device of claim 1, wherein the roosting panel defines a first opening to support a food element.

8. An insect release device, comprising:
a cylindrical container enclosing an inner volume and defining:
a container opening at a first end of the cylindrical container; and
a first port on a lateral side of the cylindrical container;
a lid to releasably engage with the container to cover the container opening and enclose the inner volume, the lid comprising:
a second port defined within a central region of the lid, the second port sized to interface with an insect sorting device to load insects from the insect sorting device;
a cylindrical member positioned to slidably interface with the second port and extend to a second end of the cylindrical container;
a roosting structure within the cylindrical container, the roosting structure comprising:
an end support comprising a shape matching an interior of the cylindrical container;
at least two roosting panels extending vertically along a length of the cylindrical container from the end support and also extending radially from the center of the cylindrical container, the at least two roosting panels and the cylindrical member dividing the inner volume into at least two distinct volumes; and
a rotation hub extending from the end support along the length of the cylindrical container and sized to extend through the second port when the roosting structure is within the inner volume enclosed by the cylindrical container and the lid, enabling rotation of the roosting structure within the cylindrical container; and
a release cap shaped to releasably seal the first port.

9. The insect release device of claim 8, wherein the at least two roosting panels each comprise a textured surface for insects to grasp onto.

10. The insect release device of claim 8, wherein the roosting structure within the inner volume rotates with respect to the cylindrical container when the rotation hub is rotated outside of the inner volume.

11. The insect release device of claim 10, wherein the first port provides selective access to the at least two distinct volumes depending on a rotational position of the roosting structure within the inner volume.

12. The insect release device of claim 8, wherein the cylindrical member comprises a hollow cylinder.

13. The insect release device of claim 8, wherein a plurality of holes is defined in the roosting panels, each hole having a diameter smaller than a dimension of an insect.

14. The insect release device of claim 8, wherein the roosting structure further comprises a bottom support coupled to ends of the at least two roosting panels and parallel to the end support.

15. An insect release device, comprising:
a cylindrical container defining an inner volume with an opening at a first end; and
a lid to releasably engage with the cylindrical container to cover the opening and enclose the inner volume, the lid comprising;
a perimeter portion; and
a rotating panel rotatably coupled to the perimeter portion positioned to rotate independently of the perimeter portion, the rotating panel comprising:
a first port positioned within a central portion of the lid;
a second port positioned within an off-center portion of the lid;
a cylindrical member positioned to slidably interface with the first port and extending through the inner volume to a second end of the cylindrical container; and
a roosting structure, comprising:
an end support having a circular shape; and
at least two roosting surfaces coupled to the end support and positioned to extend into the inner volume, the at least two roosting surfaces and the cylindrical member dividing the inner volume into at least two distinct volumes.

16. The insect release device of claim 15, wherein the at least two roosting surfaces each comprise a textured surface.

17. The insect release device of claim 15, wherein the second port provides access to each of the at least two distinct volumes as the rotating panel is rotated within the cylindrical container.

18. The insect release device of claim 17, wherein an inner wall of the cylindrical container is textured for insect roosting.

19. The insect release device of claim 15, further comprising a release cap shaped to releasably seal the second port.

20. The insect release device of claim 15, further comprising a second rotating panel rotatably coupled to the lid and comprising a third port positioned to provide access to the inner volume only when the second and third port are rotatably aligned.

21. The insect release device of claim 15, wherein the cylindrical container defines a third port at the second end with a releasable connection at a perimeter of the second opening such that the cylindrical container defines a conduit from the second port to the third port.

22. The insect release device of claim 21, further comprising:
a second lid shaped to close the cylindrical container at the second opening, the second lid comprising;
an edge comprising a releasable connection that releasably secures the second lid to the cylindrical container at the second opening and encloses the inner volume; and
a rotating panel rotatably coupled to the edge positioned to rotate independently of the edge, the rotating panel defining a release port positioned at an off-center portion of the lid.

23. An insect release device comprising:
a primary chamber defining an inner volume, the primary chamber comprising:
a first port shaped to interface with an insect sorting device and enable loading insects from the insect sorting device and into the primary chamber;
two chamber ports defined by the primary chamber, each defining a first conduit between an exterior and the inner volume; and
a slot defined between the two chamber ports defining a second conduit between the exterior and the inner volume;
a divider comprising an extension positioned to slidably insert into the slot and extend a length of the primary chamber to divide the inner volume into two distinct volumes when the divider is inserted into the slot; and
two secondary chambers positioned to slidably couple with the two chamber ports to enclose the two distinct volumes within the two secondary chambers.

24. The insect release device of claim 23, wherein the two secondary chambers each seal an opening of one of the two secondary chambers when fully inserted into the two distinct volumes.

25. The insect release device of claim 23, wherein the primary chamber comprises a rectangular block defining the two chamber ports on an opposite end of the first port.

26. The insect release device of claim 23, further comprising:
a third chamber port defined by the primary chamber adjacent one of the two chamber ports;
a second slot defined between the third chamber port and one of the two chamber ports;
a third secondary chamber positioned to slidably couple with the third chamber port; and
wherein:
the slot is a first slot; and
the divider comprises a second extension which slidably inserts into the second slot and extends the length of the primary chamber such that, when inserted into the first slot and the second slot, the divider divides the inner volume into three distinct volumes.

27. The insect release device of claim 23, wherein an interior surface of each of the two secondary chambers is textured for insect roosting.

28. The insect release device of claim 23, wherein each of the secondary chambers comprises a roosting surface which extends along a length of each respective secondary chamber.

29. A method of releasing insects, comprising:
inserting a plurality of roosting surfaces into a container of an insect release device;
securing a lid to the container, thereby enclosing an inner volume with the plurality of roosting surfaces inside the inner volume;
loading insects into the insect release device from an insect sorting device through an introduction port;
sealing the introduction port of the release device; and
opening a release port of the release device to release the insects.

30. The method of claim 29, wherein the introduction port comprises the release port.

31. The method of claim 29, wherein the introduction port is positioned on the lid of the container and the release port is positioned on a side wall of the container.

32. The method of claim 29, wherein sealing the introduction port comprises inserting a cylinder through the introduction port after loading the insects, the cylinder blocking the introduction port and mating with the plurality of roosting surfaces to divide the inner volume into separate volumes.

33. The method of claim 32, wherein opening the release port of the release device comprises opening the release port on a side of the container to release insects from one of the separate volumes.

34. The method of claim 33, further comprising rotating the plurality of roosting surfaces to release insects from a second one of the separate volumes through the release port.

35. The method of claim 32, wherein opening the release port of the release device comprises opening the release port on an off-center portion of the lid to release insects from one of the separate volumes.
